(19) United States
(12) Patent Application Publication
(10) Pub. No.: US 2026/0165636 A1
(43) Pub. Date: Jun. 18, 2026

(54) Methods for Optimizing Evoked Potential Baseline Reference Value in an Intraoperative Neurophysiological Monitoring Environment

(71) Applicant: Cadwell Laboratories, Inc., Kennewick, WA (US)

(72) Inventors: Julia Aina Caviness, Collegeville, PA (US); Michael Batdorf, Benton City, WA (US); Joseph Callahan Myers, Kennewick, WA (US); John A. Cadwell, Richland, WA (US)

(21) Appl. No.: 19/421,313

(22) Filed: Dec. 16, 2025

Related U.S. Application Data
(60) Provisional application No. 63/735,249, filed on Dec. 17, 2024.

(51) Int. Cl. A61B 5/00

(52) U.S. Cl. CPC A61B 5/4047; A61B 5/6814; A61B 5/7267; A61B 5/7271

(57) ABSTRACT

A method of determining an optimized stimulus intensity required to obtain an evoked potential (EP) response from a patient in a surgical intraoperative neurophysiological monitoring environment includes determining an initial stimulus current, determining an initial count to determine a predefined signal-to-noise ratio (SNR) threshold, and using a search algorithm for automatically generating an optimized/baseline stimulus intensity and average count required to elicit a maximal EP response from the patient based on the initial stimulus current and the initial count.

### CROSS-REFERENCE

The present application relies on U.S. patent Provisional application Ser. No. 63/735,249, titled “Methods for Optimizing Evoked Potential Baseline Reference Value in an Intraoperative Neurophysiological Monitoring Environment” and filed on Dec. 17, 2024, for priority, which is herein incorporated by reference in its entirety.

### FIELD

The present specification is related generally to the field of neurophysiological stimulation. More specifically, the present specification is related to methods for optimizing an evoked potential baseline reference value in an intraoperative neurophysiological monitoring environment.

### BACKGROUND

Intraoperative neurophysiological monitoring (IONM) is directed towards identifying, mapping and monitoring neural structures in accordance with their functions with a goal of preserving the structural integrity of these neural structures during physically invasive procedures such as surgery. Intraoperative monitoring of nerves and the spinal cord are an important part of neurosurgical and orthopedic procedures, and specifically, spinal surgeries, where the monitoring provides guidance to the surgeon on the state of the nerves and allows procedural changes which may mitigate injury to the nerve.

Monitoring both the sensory pathways and the motor pathways are important, but the technical requirements are different for the two cases. The motor response from nerve stimulation can be obtained from a single stimulus. The motor response from cortical stimulation is more complex but can also be obtained from one or a few stimuli. The sensory nerves are monitored by electrically stimulating a peripheral nerve of a patient, often at the wrist, knee, or ankle, and recording the resulting brain activity from the sensory cortex of the patient. The response is called the SomatoSensory Evoked Potential (SSEP).

FIG. 1A is a pictorial depiction of stimulus provided to a patient and which results in the generation of corresponding response waveforms recorded in an IONM system. In a typical IONM system, a stimulus 150 is provided to a patient 152 via one or more stimulating electrodes 154 coupled and positioned in proximity to a peripheral nerve of the patient 152. The stimulus 150 causes a neurological response 156, which is collected via one or more pick-up electrodes 158 coupled and positioned in proximity to the patient's sensory cortex and is displayed as waveforms 160 on a display instrument 162 of the IONM system.

The SSEP responses generated by the patient's brain in response to stimuli provided to the patient's peripheral nerves are small, and in addition, comprise significant electrical noise from sources such as, but not limited to, electrode noise, anesthesia effects, electroencephalogram (EEG) signals, muscle signals, other noise sources and other stimulating signals causing the patient's brain to produce a variety of stochastic responses. Signal processing techniques are required to improve the signal to noise ratio (SNR) of the response signals and achieve a repeatable response.

Additionally, a baseline stimulus intensity value is required to compare with real time patient responses during surgery. However, determining an optimal baseline stimulation level and signal processing parameters for both the initial and subsequent monitoring is currently tedious, time consuming and potentially suboptimal. Usually, the time required to determine the parameters required for determining an optimal baseline stimulation intensity is on the order of 5 to 10 minutes. In some cases, a very low stimulus intensity is used to achieve a supramaximal response leading to a suboptimal solution, while in other cases more than required stimulation intensity may be used which may lead to injuries in the patient's skin. Further, the technicians qualified to determine optimal stimulation parameters may not be available when required, such as during surgery.

Hence, there is need for automated systems and methods that control stimulation and data acquisition from a patient to determine a minimum stimulus intensity and the number of averages (average count) required to elicit a maximal EP response from the patient and for generating an optimized EP baseline reference value specific to the patient.

### SUMMARY

The following embodiments and aspects thereof are described and illustrated in conjunction with systems, tools and methods, which are meant to be exemplary and illustrative, and not limiting in scope. The present application discloses numerous embodiments.

In some embodiments, the present specification describes a computer-implemented method for determining an initial stimulus intensity and averaging count that elicit an evoked potential (EP) response amplitude from a patient within a region of a stimulus response curve as input to a later second stage, the method comprising: determining a baseline noise level by acquiring neurological data for one or more selected peripheral nerves of the patient, wherein the neurological data is generated in an absence of applied peripheral electrical stimulation; computing, based on the baseline noise level, an initial count representing a function of a number of stimulus trials; stimulating said one or more peripheral nerves of the patient over the initial count using an initial stimulation current, wherein the initial stimulation current is selected from within a region of a stimulus-response curve; acquiring EP response data over the initial count and determining an EP response amplitude associated with the initial stimulation current; and determining whether the EP response amplitude is indicative of a valid EP response amplitude within the region, wherein, if a valid EP response amplitude is not obtained, incrementally increasing at least one of the initial count or the initial stimulation current and iterating the steps of acquiring EP response data and determining whether the EP response amplitude is indicative of a valid EP response amplitude within the region, and wherein, if either a valid EP response amplitude is obtained or when predefined limits on increasing the initial count or the stimulation current are reached, terminating the method.

Optionally, the initial count represents a function of a number of stimulus trials to be averaged to achieve a predefined level of signal-to-noise ratio (SNR). Still optionally, the predefined level of signal-to-noise ratio is at least 2:1.

Optionally, the validity of the EP response amplitude is determined by performing a peak-to-peak calculation of the acquired EP response data using a peak detection algorithm.

Optionally, incrementally increasing the initial count comprises successively doubling the number of stimulus trials until the predefined limit corresponding to the initial count is reached.

Optionally, the region corresponds to a segment of the stimulus-response curve in which changes in stimulus current produce substantially linear changes in EP response amplitude.

Optionally, the method further comprises performing an additional search using the initial starting stimulus current and amplitude from the initial search to generate valid data points on a patient-specific stimulus-response curve, the additional search comprising: stimulating said one or more peripheral nerves using a plurality of stimulus intensities to generate stimulus-response data points, wherein the plurality of stimulus intensities range from approximately thirty percent below to approximately thirty percent above the stimulation current corresponding to the maximal EP response amplitude; determining peak-to-peak amplitudes of resulting EP response waveforms at each of the plurality of stimulus intensities; rejecting the stimulus-response data points exhibiting SNR less than or equal to a predefined threshold and accepting the stimulus-response data points exhibiting SNR above the predefined threshold; performing a linear regression over the accepted stimulus-response data points to construct a linear approximation of the patient-specific stimulus-response curve; performing a nonlinear curve fit over the accepted stimulus-response data points to determine an optimal stimulation current corresponding to a maximal EP response amplitude; and acquiring a high average-count EP response at the optimal stimulation current to establish an optimized EP baseline reference waveform for the patient.

Optionally, the additional search comprises acquiring between three and eight stimulus-response data points. Optionally, the nonlinear curve fit is performed using a logistic model.

Optionally, the high average-count response acquired to establish the optimized EP baseline reference waveform comprises at least 100 average counts.

In some embodiments, the present specification discloses a system for determining a stimulus intensity and averaging count that elicit a maximal evoked potential (EP) response amplitude from a patient, the system comprising: an EP stimulus optimizer comprising at least one stimulating electrode positioned in proximity to a peripheral nerve of the patient and one or more electrodes positioned on the patient's head in proximity to the sensory cortex; a computing device in data communication with the EP stimulus optimizer and comprising at least one processor and a non-volatile memory for storing a plurality of instructions of programmatic code which, when executed, configure the processor to: acquire neurological data, for the peripheral nerve of the patient, in the absence of peripheral stimulation to determine a baseline noise level; compute, based on the baseline noise level, an initial count representing a function of a number of stimulus trials; stimulate the peripheral nerve of the patient over the initial count using an initial stimulation current, wherein the initial stimulation current is selected from within a region of a modeled stimulus-response curve; acquire EP response data over the initial count and determine an EP response amplitude associated with the initial stimulation current; and determine whether the EP response amplitude is indicative of a valid EP response amplitude within the region, wherein, if a valid EP response amplitude is not obtained then incrementally increase at least one of the initial count or the stimulation current and iterate the steps of acquiring EP response data and of determining if the EP response data is indicative of a valid EP response amplitude within the region, and wherein, if either a valid EP response amplitude is obtained or predefined limits are reached on increasing the initial count or the stimulation current, terminate the execution of the plurality of instructions of programmatic code.

Optionally, the initial count represents a function of a number of stimulus trials to be averaged to achieve a predefined level of signal-to-noise ratio (SNR).

Optionally, the predefined level of signal-to-noise ratio is at least 2:1.

Optionally, the validity of the EP response amplitude is determined by performing a peak-to-peak calculation of the acquired EP response data using a peak detection algorithm.

Optionally, incrementally increasing the initial count comprises successively doubling the number of stimulus trials until the predefined level corresponding to the initial count is reached.

Optionally, the region corresponds to a segment of the modeled stimulus-response curve in which changes in stimulus current produce substantially linear changes in EP response amplitude.

Optionally, the plurality of instructions of programmatic code further configure the processor to perform an additional search to generate valid data points on a patient-specific stimulus-response curve, the additional search comprising: stimulating said one or more peripheral nerves using a plurality of stimulus intensities to generate stimulus-response data points, wherein the plurality of stimulus intensities range from approximately thirty percent below to approximately thirty percent above the stimulation current corresponding to the maximal EP response amplitude; determining peak-to-peak amplitudes of resulting EP response waveforms at each of the plurality of stimulus intensities; rejecting the stimulus-response data points exhibiting SNR up to and including a predefined threshold and accepting the stimulus-response data points exhibiting SNR above the predefined threshold; performing a linear regression over the accepted stimulus-response data points to construct a linear approximation of the patient-specific stimulus-response curve; performing a nonlinear curve fit over the accepted stimulus-response data points to determine an optimal stimulation current corresponding to a maximal EP response amplitude; and acquiring a high average-count EP response at the optimal stimulation current to establish an optimized EP baseline reference waveform for the patient.

Optionally, the additional search comprises acquiring between three and eight stimulus-response data points.

Optionally, the nonlinear curve fit is performed using a logistic model.

Optionally, the high average-count response acquired to establish the optimized EP baseline reference waveform comprises at least 100 average counts.

In some embodiments, the present specification discloses a computer-implemented method for determining a stimulus intensity and averaging count that elicit a maximal evoked potential (EP) response amplitude from a patient, the method comprising: acquiring neurological data, for one or more selected peripheral nerves of the patient, in the absence of peripheral stimulation to determine a baseline noise level; computing, based on the baseline noise level, an initial count representing a function of a number of stimulus trials that are averaged to achieve a predefined level of signal-to-noise ratio (SNR); stimulating said one or more peripheral nerves of the patient over the initial count using a stimulation current and acquiring EP response data over the initial count, wherein the stimulation current is selected from within a linear region of a modeled stimulus-response curve; iteratively adjusting the initial count and the stimulation current until a valid EP response amplitude is obtained, thereby determining stimulation current and an average count corresponding to the valid EP response amplitude; stimulating the one or more peripheral nerves using a plurality of stimulus intensities to generate stimulus-response data points, wherein the plurality of stimulus intensities range from approximately thirty percent below to approximately thirty percent above the stimulation current corresponding to the valid EP response amplitude; determining peak-to-peak amplitudes of resulting EP response waveforms at each of the plurality of stimulus intensities; rejecting the stimulus-response data points exhibiting SNR up to and including the predefined threshold and accepting the stimulus-response data points exhibiting SNR above the predefined threshold; performing a linear regression over the accepted stimulus-response data points to construct a linear approximation of the patient-specific stimulus-response curve; performing a nonlinear curve fit over the accepted stimulus-response data points to determine an optimal stimulation current corresponding to the maximal EP response amplitude; and acquiring a high average-count EP response at the optimal stimulation current to establish an optimized EP baseline reference value specific to the patient.

Optionally, the nonlinear curve fit is performed using a logistic model.

Optionally, the high average-count response acquired to establish the optimized EP baseline reference value comprises at least 100 average counts.

The aforementioned and other embodiments of the present specification shall be described in greater depth in the drawings and detailed description provided below.

### BRIEF DESCRIPTION OF THE DRAWINGS

The accompanying drawings illustrate various embodiments of systems, methods, and embodiments of various other aspects of the disclosure. Any person with ordinary skill in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1A is a pictorial diagram showing the flow of stimulus data delivered to a theoretical patient and corresponding response waveforms recorded in a conventional IONM system;

FIG. 1B is a block diagram illustrating a flow of data among a plurality of components of an EP stimulus optimizer, in accordance with embodiments of the present specification;

FIG. 2 illustrates an exemplary stimulus response curve, generated by using the system of the present specification, in embodiments;

FIG. 3 is a flow chart detailing steps of a method for performing an initial search for obtaining a desired response waveform in an EP stimulus optimizer, in accordance with an embodiment of the present specification;

FIG. 4 is a flow chart detailing steps of a method for performing a detailed search for determining valid data points on a stimulus response curve for obtaining an optimized EP baseline reference value specific to the patient, in accordance with an embodiment of the present specification;

FIG. 5A is a stimulus response curve plot generated by using a three straight segment approximation, in accordance with an embodiment of the present specification;

FIG. 5B is a stimulus response curve plot generated by using a logistic function, in accordance with an embodiment of the present specification; and

FIG. 6 is a flow chart detailing steps of a method for determining an optimized stimulus intensity required to obtain an evoked potential (EP) response from a patient in a surgical intraoperative neurophysiological monitoring environment, in accordance with an embodiment of the present specification.

### DETAILED DESCRIPTION

In embodiments, the present specification describes an evoked potential (EP) stimulus optimizer. In various embodiments, the present specification is directed towards a method for automatically determining a minimum and/or optimal stimulus intensity that elicits a full SomatoSensory Evoked Potential (SSEP) response from a patient to determine a baseline signal, and for providing feedback to a surgeon and/or technician whether and if the stimulation intensity and baseline are optimized. The systems and methods of the present specification support surgeon directed monitoring, wherein the surgeon is responsible for placing electrode(s) on a patient's body, stimulating the patient via the electrodes, and interpreting the monitored patient response data, without the help of a monitoring technician.

In embodiments, the present specification provides an automated algorithm that controls both stimulation and data acquisition from a patient to determine a minimum stimulus intensity and average count required to elicit a maximal EP response from the patient, for generating an optimized EP baseline reference value specific to the patient. Also provided are quality metrics gained from the optimization process and feedback to the user regarding the status of the stimulus optimization process.

In various embodiments, the average count represents the number of sequential stimulation attempts that are averaged together to obtain a desired response signal having a high SNR and minimal signal noise. Signal noise decreases by a proportion of 1/(square root of the average count), whereby doubling the average count would reduce noise by 30%.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In the description and claims of the application, each of the words “comprise”, “include”, “have”, “contain”, and forms thereof, are not necessarily limited to members in a list with which the words may be associated. Thus, they are intended to be equivalent in meaning and be open-ended in that an item or items following any one of these words is not meant to be an exhaustive listing of such item or items, or meant to be limited to only the listed item or items. It should be noted herein that any feature or component described in association with a specific embodiment may be used and implemented with any other embodiment unless clearly indicated otherwise.

It should also be noted that as used herein and in the appended claims, the singular forms “a,” “an,” and “the” include plural references unless the context dictates otherwise. Although any systems and methods similar or equivalent to those described herein can be used in the practice or testing of embodiments of the present disclosure, the preferred systems and methods are now described.

In various embodiments, a computing device includes an input/output controller, at least one communications interface and system memory. The system memory includes at least one random access memory (RAM) and at least one read-only memory (ROM). These elements are in communication with a central processing unit (CPU) to enable operation of the computing device. In various embodiments, the computing device may be a conventional standalone computer or alternatively, the functions of the computing device may be distributed across multiple computer systems and architectures.

In some embodiments, execution of a plurality of sequences of programmatic instructions or code enable or cause the CPU of the computing device to perform various functions and processes. In alternate embodiments, hard-wired circuitry may be used in place of, or in combination with, software instructions for implementation of the processes of systems and methods described in this application. Thus, the systems and methods described are not limited to any specific combination of hardware and software.

The term “module”, “application”, “component” or “engine” used in this disclosure may refer to computer logic utilized to provide a desired functionality, service or operation by programming or controlling a general-purpose processor. Stated differently, in some embodiments, a module, application, or engine implements and/or is configured to implement a plurality of instructions or programmatic code to cause a general-purpose processor to perform one or more functions. In various embodiments, a module, application or engine can be implemented in hardware, firmware, software or any combination thereof. The module, application or engine may be interchangeably used with unit, logic, logical block, component, or circuit, for example. The module, application or engine may be the minimum unit, or part thereof, which performs one or more particular functions. It should be noted herein that each hardware component is configured to perform or implement the plurality of instructions or programmatic code to which it is associated, but not limited to such functions.

The term “maximal EP response” as used in this disclosure may refer to the largest amplitude of an evoked potential waveform described as when the waveform amplitude plateaus as stimulus intensity is incrementally increased, representing a condition where further increases in stimulus intensity produce no substantial increase in response amplitude, on the order of 1%-3%, thereby indicating full recruitment of responsive neural fibers.

The term “peripheral electrical stimulation” as used in this disclosure may refer to a controlled application of electrical current to a patient's peripheral nervous system, i.e. to one or more nerves located outside the brain and spinal cord, using surface or subdermal electrodes. The purpose of such stimulation is to evoke a reproducible electrophysiological response, such as a somatosensory or motor evoked potential (EP), that can be detected by recording electrodes.

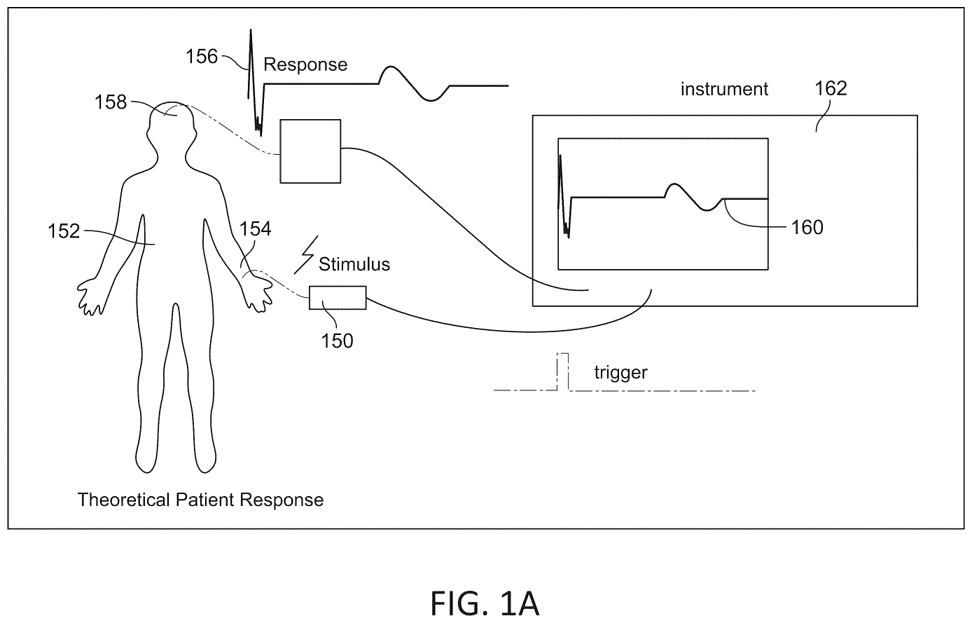

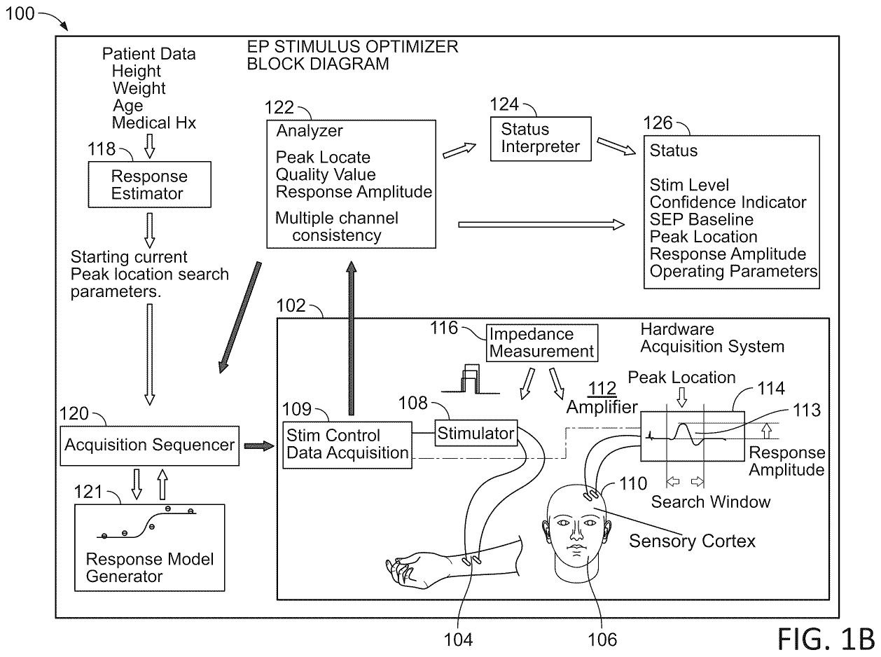

FIG. 1B is a block diagram illustrating a flow of data among a plurality of components of an EP stimulus optimizer, in accordance with an embodiment of the present specification. EP stimulus optimizer 100 comprises a hardware acquisition system 102 comprising stimulating electrodes 104 positioned in proximity to a peripheral nerve of a patient 106, such as near the patient's wrist, knee or ankle. While two electrodes 104 are shown in FIG. 1B as connected to the patient's 106 wrist, a plurality of electrodes may be connected to a plurality of areas on the patient's body in proximity to the patient's peripheral nerves in order to obtain a stronger neurological response for generating an optimized EP baseline reference value specific to the patient.

The peripheral nerve of the patient 106 is electrically stimulated via the electrodes 104 by using a stimulator 108 and the resulting brain activity, i.e. neurological response is obtained via pick-up electrodes 110 placed on the patient's 106 head, in proximity to the patient's sensory cortex. The obtained neurological response is amplified by an amplifier 112 and displayed as waveforms 113 on a display system 114. An impedance measurement module 116 is configured to measure impedance of one or both of the stimulating electrodes 104 and the pick-up electrodes 110 in order to verify a required level of electrode application and positioning. The impedance measurement module 116 ensures that the application and position of the electrodes is in order for the impedance level of said electrodes to be in the requisite range. In embodiments, an impedance level of the stimulating electrodes 104 and the pick-up electrodes 110 is required to be below 5 kOhms for operation of the EP stimulus optimizer 100.

In embodiments, the hardware acquisition system 102 is in data communication with a computing device that executes a plurality of instructions or programmatic code associated with modules or engines such as, for example, a stimulation control data acquisition module 109, a response estimator module 118, an acquisition sequencer module 120, a response model generator module 121, an analyzer module 122, and a status interpreter module 124.

EP stimulus optimizer 100 further comprises a response estimator module 118 which is configured to receive a plurality of patient parameters as input, for determining an initial stimulation response curve corresponding to the patient 106. In embodiments, the patient parameters comprise one or more of height, weight, age, and medical history of any underlying diseases/conditions. In an embodiment, the patient parameters are manually entered into the response estimator module 118 prior to commencement of the optimizer 100 operation. The entered data is stored in, and correlated with, an existing database in order to optimize the operation of the EP stimulus optimizer 100.

The initial stimulation response curve corresponding to the patient 106 provided by the response estimator module 118 comprises a starting current and pulse width for stimulation, the total number of stimulus pulses to average, and search parameters for locating a peak stimulation current. These are based on known normal values for the site being evaluated. For example, for an upper extremity SSEP the starting values may be on the order of 100 usec 15 mA pulses, 100 averages, and a latency search centered around 20 msec.

The stimulator 108 is configured to be controlled by a stimulation control data acquisition module 109 which, in turn, is configured to receive input from an acquisition sequencer module 120. Initially, the acquisition sequencer module 120 is configured to cause the stimulation control data acquisition module 109 to stimulate the patient 106 averaging at zero intensity in order to establish a baseline SNR and a number of averages (average count) needed to start the stimulation.

The acquisition sequencer module 120 is also in communication with a response model generator module 121. The number of stimulus pulses to arrive at an average intensity of the pulses are varied within a predefined limit by the acquisition sequencer module 120, which, along with the response model generator module 121, is configured to generate valid data for analysis. In embodiments, the predefined limit within which the number of the stimulus pulses is varied to arrive at an average intensity is defined by a minimum number of pulses needed to elicit a muscle twitch in the patient and a maximum number of pulses that does not cause excessive patient movement and tissue damage. The maximum and minimum number of stimulus pulses, as defined above, are dependent upon a plurality of variables such as, but not limited to patient pathology/age, technical setup of the optimizer 100, distance between a stimulus and a target nerve, and level of anesthesia given to the patient.

In an embodiment, a non-stimulating signal to noise ratio (SNR) of the optimizer 100, as well as observational studies may be used to determine the minimum and maximum number of stimulus pulses. During use of the optimizer 100 in an operation theater where the patient has been administered a muscle relaxant that may mask the muscle twitch, the peripheral response of the patient to stimulus pulses is used to determine the minimum and maximum number of stimulus pulses corresponding to the patient, as peripheral responses are least affected by anesthetic agents. In embodiments, the valid data for analysis is generated by the response model generator module 121 based upon a range of minimum and maximum amplitude of response and noise, corresponding to the patient 106. In an embodiment, the valid data for analysis is obtained within an expected response window which may be predetermined based on one or more of the patient's pathology, age, weight or height.

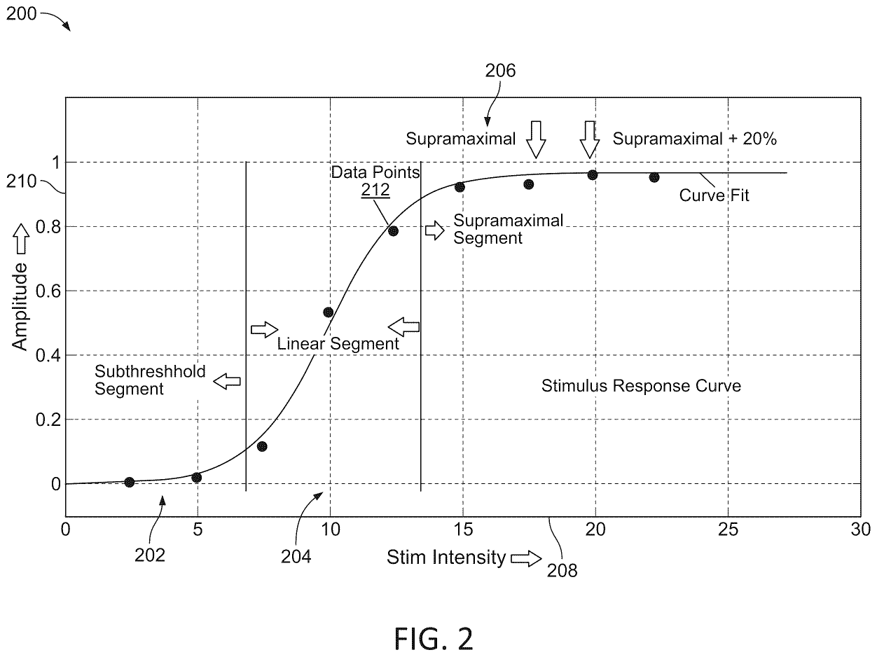

FIG. 2 illustrates an exemplary stimulus response curve, in accordance with an embodiment of the present specification. For the purposes of computing an optimized EP baseline reference value specific to a patient, the response model generator module 121 is configured to divide the stimulus response curve 200 into a subthreshold segment 202, a linear segment or region 204 and a supramaximal segment 206. In an embodiment, the response model generator module 121 divides the stimulus response curve 200 corresponding to predefined segmentation values which may be based upon the patient's demographics. Stimulation intensity is represented along the X-axis 208 of the response curve 200, while the Y-axis 210 represents amplitudes of corresponding neurological response waveforms obtained as a result of the corresponding stimulation. Thus, the response curve 200 is generated by plotting the stimulation intensities provided to the patient and the amplitude of the corresponding response waveform obtained, providing a plurality of data points 212. As shown in FIG. 2, in subthreshold segment 202, there is no response across multiple stimulation intensities. In linear segment 204, the amplitude of response waveform increases with increasing stimulation intensity. In supramaximal segment 206, the amplitude remains substantially constant across multiple stimulation intensities.

Referring back to FIG. 1B, the stimulation control data acquisition module 109 is in communication with an analyzer module 122 which is configured to compute parameters of the response waveform 113 such as, but not limited to, location of the response peak, quality value and amplitude of the response, and consistency of the response over multiple channels. In embodiments, analyzer module 122 is a dense input analyzer that is configured to perform heuristic evaluation of responses for validity and consistency. The analyzer module 122 is configured to provide the computed parameters as feedback to the acquisition sequencer module 120 which, in turn, is configured to modify the input to the stimulation control data acquisition module 109 based on the received response parameters.

In embodiments, operation of the response model generator module 121 is initiated after the acquisition sequencer module 120 has caused the stimulation control data acquisition module 109 to stimulate the patient 106 averaging at zero intensity, in order to establish a baseline SNR and a number of averages (average count) needed to start the stimulation. If the background noise of the averaged patient response acquired during the averaging process of the first (lowest) intensity is larger than a given threshold, feedback is sent to the acquisition sequencer module 120 and the process for the corresponding channel is aborted.

The analyzer module 122 is in communication with a status interpreter module 124 which is configured to receive input from the analyzer module 122 and further configured to generate status data 126 comprising a stimulation level, confidence indicator, an SEP baseline level, a location of response peak, an amplitude of the response waveform, and other operating parameters corresponding to the EP stimulus optimizer 100. In embodiments, the status interpreter module 124 is configured to generate human understandable explanations about the validity and limitations of the results obtained by the analyzer module 122 and is also configured to provide suggestions for technical changes if required.

In embodiments, the collection of data points for the EP Stimulus Optimizer 100 is started manually from a control screen (not shown) that is in communication with the stimulation system. In an embodiment, all EP modes that use an algorithm corresponding to the EP stimulus optimizer 100 are run concurrently. In an embodiment, the EP modes comprise an upper SSEP mode and a lower SSEP mode. A user may select participating modes and channels from a settings dialog provided via an EEG control screen. Upon completion or termination of the EP stimulus optimizer 100 search for obtaining a desired response waveform fit, a status of the search is reported to the user and determined optimized EP baseline reference value are displayed. In embodiments, a search/fit is successful if data input to one or more linear and later non-linear logistics models generally follows the trend of the logistic models. In an embodiment, the fit is measured using adjustable thresholds determined by experiment. If a fit is not obtained, the search is deemed a failure for the corresponding channel. If the search was not successful, EP stimulus optimizer 100 is terminated, and the reason for termination is specified. In embodiments, human understandable text is displayed to the user describing the failure. In embodiments, results obtained from the EP stimulus optimizer 100 are stored along with corresponding patient records.

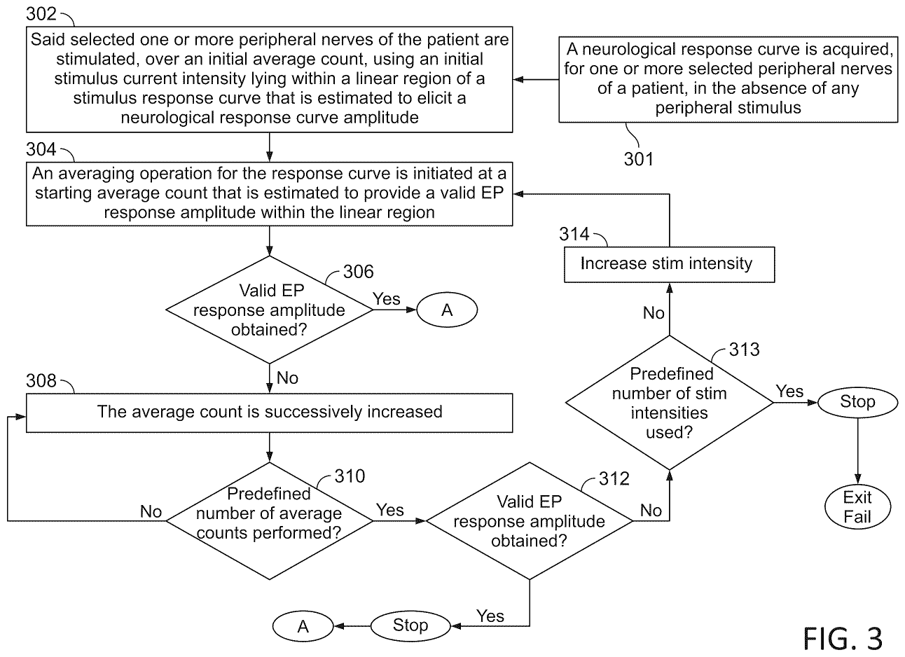

FIG. 3 is a flow chart detailing a method for obtaining a desired evoked potential (EP) response waveform that will provide the starting intensity for a later detailed search of intensity in an EP stimulus optimizer, in accordance with an embodiment of the present specification. In embodiments, the computing device, in data communication with the hardware acquisition system 102 (FIG. 1B), executes the method of FIG. 3.

At step 301, a neurological response curve (or neurological data) is acquired, for one or more selected peripheral nerves of a patient, in the absence of any peripheral stimulus. In embodiments, the neurological data collected at this stage represents only passive neurological and environmental noise, the characteristics of which are dependent on the patient and the surrounding environment. The acquisition of this passive neurological and environmental noise is used to establish a baseline noise level (or baseline SNR) that is used to define an initial average count for subsequent stimulus intensity increments, based on that noise. The initial average count a) is a minimum number of stimulus trials that must be averaged to ensure that random noise fluctuations (or SNR) fall below a predefined noise threshold, thereby enabling clearer visualization and detection of clinically meaningful evoked responses and b) is based on the established background noise level specific to the patient and channel. In some embodiments, a minimum desired SNR is 2:1.

At step 302, the selected one or more peripheral nerves of the patient are stimulated, over the initial average count, using an initial stimulus current intensity lying within the linear region (that is, a region where changes in stimulus current are expected to produce substantially linear changes in response amplitude) of the stimulus-response curve 200 (shown in FIG. 2) that is estimated to elicit a neurological response curve amplitude. In embodiments, the stimulation current intensity lies within a range of 10-20 mA for healthy patients. In some embodiments, the stimulus-response curve 200 (shown in FIG. 2) is based on a plurality of patient parameters such as, for example, one or more of the patient's height, weight, age, and medical history of any underlying diseases/conditions. Since the stimulus-response curve is specific to a population of individuals, accounting for the plurality of patient parameters supports the modeled stimulus-response curve 200 in reflecting variations in expected waveform amplitude latencies, ensuring identification of appropriate evoked-potential peak corresponding to the physiological response being monitored. Such adjustments allow correct determination of the maximal EP response amplitude even in patients whose waveform characteristics, particularly latency, may fall outside standard normative ranges, such as for tall individuals, obese patients, geriatric patients, or patients with pre-existing neurological or systemic conditions.

At step 304, an averaging operation for the response curve is initiated at a starting average count that is estimated to provide a valid EP response amplitude within the linear region that is several standard deviations above the baseline. In some embodiments, the starting average count is the initial average count of step 301. Alternatively, in some embodiments, the starting average count is a scaled multiple of the initial average count of step 301.

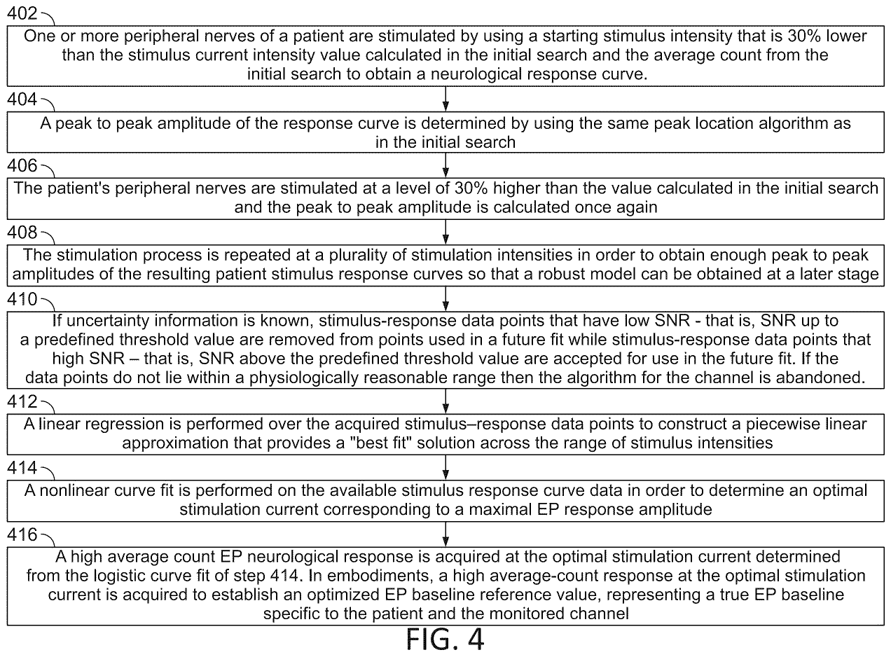

Thereafter, at step 306, a peak detection algorithm is applied to acquire EP response amplitudes, in order to determine whether a valid EP response amplitude has been obtained. It should be appreciated that a peak-to-peak amplitude calculation (using the peak detection algorithm described in step 404 of FIG. 4) determines the validity of the EP response amplitude. Thus, the system, as described with respect to step 301 records data when there is no stimulus (so that any signal that is seen is assumed to be noise) and from that non-stimulated data estimates the typical size (or standard deviation) of the noise, which can be referred to as σ. When a stimulus is applied, the EP waveform will have a maximum point (Peak) and a minimum point (trough). The peak-to-peak amplitude is simply the difference between that maximum and minimum. A response is labeled as “valid” only if its peak-to-peak amplitude is at least 2σ, preferably at least 3σ. In other words, the EP has to be at least three times larger than the typical noise fluctuations, which is a predetermined threshold that reduced the chance of falsely labeling noise as a real response.

If a valid EP response amplitude within the linear region has been obtained, the process concludes successfully for the given channel and transitions to a next stage, which, in embodiments, is the detailed search described with respect to FIG. 4. If the valid EP response amplitude within the linear region is not obtained, the method proceeds to step 308, where the average count (that is, the number of stimulus trials that must be averaged to ensure that random noise fluctuations (or SNR) fall below a predefined noise threshold) is successively increased (such as, for example, in multiples of two) in order to improve SNR and enhance detectability of the response peak. At step 310, it is determined whether a predefined number of average-count increments has been performed. If not, steps 308 and 310 are repeated. If the predefined number of average counts has been reached, the process advances to step 312, where it is again determined whether a valid EP response amplitude has been achieved using the increased averaging. If a valid EP response amplitude has been obtained, the process concludes successfully for the given channel and transitions to the next stage—that is, to the detailed search of FIG. 4. If the valid response amplitude is still not obtained, the method proceeds to step 313, where it is determined whether a predefined number of stimulus intensity levels has been used. If the predefined number of intensities has been exhausted, the process is terminated with a failure condition for that channel. However, if the predefined number of intensities has not been reached, the stimulation intensity is increased at step 314, and the method returns to step 304 to repeat the averaging sequence for the new intensity level.

Through the iterative adjustments of both the average count and stimulus current intensity, the EP stimulus optimizer automatically determines the stimulus conditions, i.e. the stimulus current intensity and the average count, necessary to elicit a valid EP response amplitude for the patient. In embodiments, once a valid EP response amplitude within the linear region has been determined by performing an initial search as shown in FIG. 3, the EP stimulus optimizer of the present specification performs a detailed search in order to determine a minimum stimulus intensity required to elicit a maximal EP response from the patient, for generating an optimized EP baseline reference value specific to the patient.

FIG. 4 illustrates a method of performing a detailed search for determining data points on a stimulus response curve for obtaining an optimized EP baseline reference value specific to a patient, in accordance with an embodiment of the present specification. At step 402, one or more peripheral nerves of the patient are stimulated by using a starting stimulus intensity that is 30% lower than the stimulus current intensity value calculated in the initial search of FIG. 3 (thereby sampling a lower, near-linear region of the patient's stimulus-response curve) and the average count from the initial search to obtain a neurological response curve. The value of 30% is chosen since an EP response typically has either a 2:1 or a 3:1 ratio between a no response segment and a supramaximal segment. Hence, decreasing the starting stimulus intensity by 30% ensures that some of the patient's stimulation response data points which are needed for determining the supramaximal segment appear in the linear segment of the response curve. It should be noted that while 30% has been selected in this embodiment, this percentage shall not be construed as limiting and any percentage may be used as long as it achieves the objectives of the present specification.

At step 404, a peak-to-peak amplitude of the response curve is determined by using the same peak detection/location algorithm as in the initial search (step 306 of FIG. 3). The peak location is at the maximum upward (negative) deflection of the average response located between a minimum latency and a maximum latency for the response. Thus, the peak detection/location algorithm first finds the maximum and minimum of the averaged EP response (for a given stimulation intensity level) over the region of interest given by the user. The algorithm is further configured to then subtract these values and calculate the resulting error. Other criteria for detection and inclusion of such a peak include its amplitude and morphology. For example, for an upper extremity adult SSEP the initial search may be in a time frame ranging from 15 to 30 msec and an amplitude greater than 1 microvolt.

At step 406, the patient's peripheral nerves are stimulated at a level of 30% higher than the stimulus current intensity value calculated in the initial search of FIG. 3, to obtain a supramaximal response, and the peak-to-peak amplitude is calculated once again.

At step 408, the stimulation process is repeated at a plurality of stimulation intensities (ranging from 3 to 8, in some embodiments) in order to obtain enough peak-to-peak amplitudes of the resulting patient stimulus response curves so that a robust model can be obtained at a later stage. In embodiments, the 3 to 8 stimulation intensities are in addition to the stimulation current intensities previously identified within the linear region during the initial search stage of FIG. 3. In some embodiments, the plurality of stimulus intensities fall in a range between approximately 30% below and 30% above the stimulation current determined during the initial search of FIG. 3. The model contemplates at least 1 supramaximal response at a known intensity, 1 supramaximal response at a higher stimulation intensity, and 1 non-response at a lower intensity to verify the shape of the stimulation intensity curve. The minimum number of intensities required is 3 sets; the maximum number is not pre-set but the search algorithm fails and terminates the stimulation when the required current exceeds recommended limits. The algorithm can also terminate successfully after 2 sets of averages if the responses at the initial stimulation intensity (which is always safe) generates a reliable supramaximal response. Intensities can lie at the midpoint between each of the previous intensities used, in addition to an intensity lying past the midpoint+30% intensity. For each stimulation intensity, a single (averaged) response curve is obtained.

In embodiments, the signal-to-noise ratio of a neurological response curve is estimated by tracking the noise characteristics obtained from the difference between successive acquired traces. As each new trace is collected during the averaging process, as configured, the system computes the difference between that trace and the immediately preceding trace, thereby subtracting out any common evoked-potential signal component and isolating primarily the noise contribution. The standard deviation of this difference waveform is then calculated to estimate the noise present in a single, non-averaged trace. Using this noise estimate, the SNR of the evoked response is determined through propagation-of-error principles.

In embodiments, multiple limb sensors may be interleaved to reduce convergence times.

After the stimulation process is completed then, at step 410, if uncertainty information or confidence measures (for example, standard deviation or variance) are known, stimulus-response data points that have low SNR up to and including a predefined threshold value are removed from data points used in a future fit while stimulus-response data points that have a high SNR above the predefined threshold value are accepted for use in the future fit. If the data points do not lie within a physiologically reasonable range then the search algorithm for the channel is abandoned.

At step 412, a linear regression is performed over the accepted stimulus-response data points to construct a linear approximation that provides a “best fit” solution across the range of stimulus intensities. In embodiments, a linear curve fit is performed to establish an initial linear model that is used both to reject unrealistic or non-physiological stimulus-response curve data and to provide initial parameter estimates for a subsequent logistic function fit. In cases where the linear approximation indicates unrealistic data, the EP stimulus optimizer search algorithm terminates or fails for that channel. The resulting model further provides an initial estimate for a more comprehensive nonlinear fitting algorithm executed at a later stage to determine the true stimulus-response characteristics.

At step 414, a nonlinear curve fit of a logistic model is performed on the available stimulus response curve in order to determine an optimal stimulation current corresponding to a maximal EP response amplitude. In embodiments, the model approximates the stimulus response curve as a sigmoid which can be divided into 3 regions, the subthreshold region, a linear region, and a supramaximal region. This continuous approximation allows defining the specific parameters of the patient's stimulus response with a minimal number of discrete stimulus response curve stimulations. Curve fitting is performed in order to automatically determine a maximal response from the acquired response data. Multiple data points near a supramaximal segment of the response curve (shown in FIG. 2) are needed for accurate determination of the maximal response and to locate the abrupt change in slope of the response curve. In embodiments, the reliability of measurement of each data point on a neurological response curve increases with increasing average count, and is adversely affected by noise in the stimulation system, which may or may not average out.

In a less preferred embodiment, a manual procedure for setting baseline stimulation intensity and acquisition parameters for neurological response curves corresponding to individual patients is performed. First, for each nerve being monitored using SEP, a stimulus level is manually selected, and an average response is obtained. Next, an automated peak detector is used to locate the response curve and measure the amplitude of the response peak. If a signal is detected, the stimulus is increased and acquisition is repeated in order to observe incremental changes in resulting response peaks, until a repeatable maximal response is found. An exemplary repeatable maximal response exhibits less than 1-3% increase in peak amplitude with a low SNR. A technician is required to run a complete set of averages and increase the stimulation current in small increments. In the manual process, lower stimulation currents and an estimate of the average count needed for obtaining a repeatable maximal response are typically not evaluated. Usually, a supramaximal response amplitude is determined by using an adequate stimulation current, after which a baseline stimulation current is determined at a higher optimal value (such as 20% higher) in order to provide a safety margin. The American Clinical Neurophysiology Society that publishes consensus statements and guidelines for the neuro-diagnostics industry recommends a stimulation current range between 10 mA to 40 mA, based on a peripheral nerve being stimulated and the corresponding patient's pathology.

However, this approach to determining a preferred stimulation current and response characteristic is not preferred and is time-consuming, more prone-to-error and may not provide a standard protocol to set or validate the parameters used. Further, the process is highly dependent on the availability and efficiency of a technician.

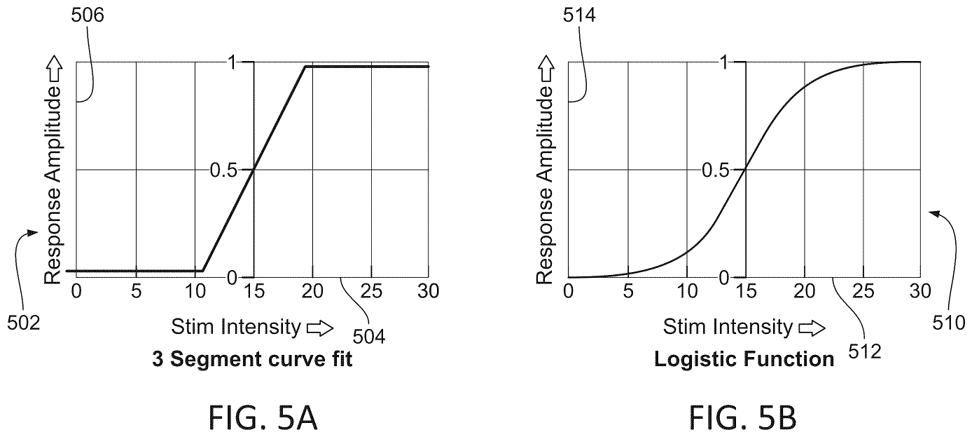

In various embodiments, a number of continuous linear approximations are used to model the stimulus response curve. FIG. 5A illustrates a stimulus response curve obtained by using a three straight segment approximation, in accordance with an embodiment of the present specification. Response curve 502 comprises three straight segments obtained by depicting stimulus intensity along x-axis 504 and response amplitude along y-axis 506.

FIG. 5B illustrates a stimulus response curve obtained by using a logistic function, in accordance with an embodiment of the present specification. Response curve 510 is obtained by using a logistic function and depicting stimulus intensity along x-axis 512 and response amplitude along y-axis 514. The logistic function is depicted as:

$$f ( x ) = \frac{L}{1 + {e}^{- k ( x - {x}_{0} )}}$$

wherein: f(x) is an output of the logistic function;
L is a maximum peak value of the response curve;
k is a logistic growth rate or steepness/slope of the response curve;
x₀ is the x value of the sigmoid midpoint or; and
x is a real number.

In embodiments, an EP stimulus optimizer search algorithm is used to fit the logistic model to the pre-collected stimulus response data points of the patient, in order to find the best-fit parameters for determining an optimal stimulus response corresponding to the patient.

In embodiments, if the search algorithm is abandoned at any point, then a failure to find an optimal stimulus response corresponding to the patient is reported along with a message describing the reason why the fit was abandoned.

In another embodiment, the logistic function described above, or a nonlinear Levenberg-Marquardt fitting algorithm is used to find the best-fit parameters for determining an optimal stimulus response corresponding to the patient. In embodiments, where the Levenberg-Marquardt fitting algorithm is used, in order to obtain a converging response curve, it is advantageous to provide: an initial estimate for the parameters, at least twice as many data points as the required parameter count, data points with a relatively low SNR, data points that cover the range of the response curve, and a higher concentration of data points in regions of a large slope in the response curve. In embodiments, the initial estimate for the parameters is obtained via the linear regression performed at step 412. In embodiments, inputs to the Levenberg-Marquardt algorithm comprise the collected data points, uncertainties for each data point, the best-guess parameters, the equation for the model used to fit the data (i.e., the logistics function), and a given tolerance to accept the fit. Uncertainties are not required, but may aid in the prioritization of low noise data collections in the fit. Results of the fit comprise the best-fit parameters and error information from the fit. The fit is accepted if the error information meets a predefined threshold, else the fit is abandoned. Using a curve fit model a stimulation channel intensity may be predefined, wherein the response curve amplitude is 99.0% of the peak value of the response curve.

At step 416, a high average count EP neurological response is acquired at the optimal stimulation current determined from the logistic curve fit of step 414. In embodiments, a high average-count response at the optimal stimulation current is acquired to establish an optimized EP baseline reference value, representing a true EP baseline specific to the patient and the monitored channel. The average count corresponding to the optimized EP baseline is high as compared to average counts used in the EP stimulus optimizer search algorithm of FIG. 3, for example, at least 100 versus 20 average counts.

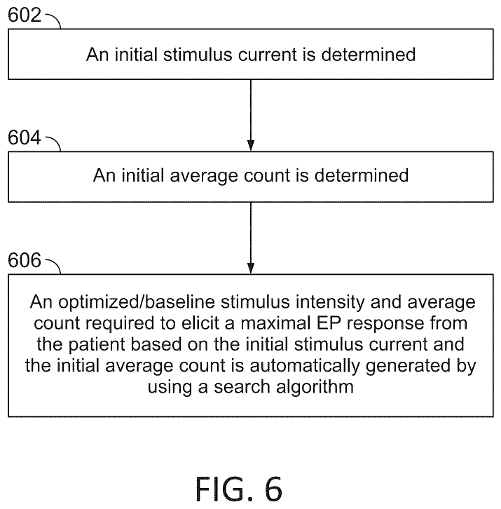

FIG. 6 is a flow chart detailing a method for determining an optimized stimulus intensity required to obtain an evoked potential (EP) response from a patient in a surgical intraoperative neurophysiological monitoring environment, in accordance with an embodiment of the present specification. At step 602, an initial stimulus current is determined. In an embodiment, determining an initial stimulus current comprises processing one or more of the patient's height, weight, age, and medical history of any underlying diseases/conditions. At step 604, an initial average count is determined. At step 606, an optimized/baseline stimulus intensity and average count required to elicit a maximal EP response from the patient based on the initial stimulus current and the initial average count is automatically generated by using the methods of the present specification as described above. In an embodiment, a response curve comprising a subthreshold segment, a linear segment and a supramaximal segment is generated; wherein no EP response is obtained across multiple stimulation intensities in the subthreshold segment, amplitude of EP response increases with increasing stimulation intensity in the linear segment, and EP response amplitude remains constant across multiple stimulation intensities in the supramaximal segment.

In an embodiment, the methods of the present specification, and in particular, the search algorithm is configured to acquire multiple valid patient data points from the generated response curve, and further configured to use each of the acquired patient data points to determine a next patient data point that is needed to converge on an optimal stimulation intensity determination. In embodiments, the method illustrated in FIG. 6 further comprises determining quality metrics of a response curve corresponding to the maximal EP response and providing feedback to a user regarding a success or failure of the search algorithm. In an embodiment, determining quality metrics of a response curve corresponding to the maximal EP response comprises three bin averaging of predefined stimulus EP responses and zero stimulation responses.

The above examples are merely illustrative of the many applications of the system of present specification. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

What is claimed is:

1. A computer-implemented method for determining an initial stimulus intensity and averaging count that elicit an evoked potential (EP) response amplitude from a patient within a region of a stimulus response curve as input to a later second stage, the method comprising:
determining a baseline noise level by acquiring neurological data for one or more selected peripheral nerves of the patient, wherein the neurological data is generated in an absence of applied peripheral electrical stimulation;
computing, based on the baseline noise level, an initial count representing a function of a number of stimulus trials;
stimulating said one or more peripheral nerves of the patient over the initial count using an initial stimulation current, wherein the initial stimulation current is selected from within a region of a stimulus-response curve;
acquiring EP response data over the initial count and determining an EP response amplitude associated with the initial stimulation current; and
determining whether the EP response amplitude is indicative of a valid EP response amplitude within the region,
wherein, if a valid EP response amplitude is not obtained, incrementally increasing at least one of the initial count or the initial stimulation current and iterating the steps of acquiring EP response data and determining whether the EP response amplitude is indicative of a valid EP response amplitude within the region, and
wherein, if either a valid EP response amplitude is obtained or when predefined limits on increasing the initial count or the stimulation current are reached, terminating the method.

2. The computer-implemented method of claim 1, wherein the initial count represents a function of a number of stimulus trials to be averaged to achieve a predefined threshold level of signal-to-noise ratio (SNR).

3. The computer-implemented method of claim 2, wherein the predefined threshold level of signal-to-noise ratio is at least 2:1.

4. The computer-implemented method of claim 1, wherein the validity of the EP response amplitude is determined by performing a peak-to-peak calculation of the acquired EP response data using a peak detection algorithm.

5. The computer-implemented method of claim 1, wherein incrementally increasing the initial count comprises successively doubling the number of stimulus trials until the predefined limit corresponding to the initial count is reached.

6. The computer-implemented method of claim 1, wherein the region corresponds to a segment of the stimulus-response curve in which changes in stimulus current produce substantially linear changes in EP response amplitude.

7. The computer-implemented method of claim 1, further comprising performing an additional search using the initial starting stimulus current and amplitude from the initial search to generate valid data points on a patient-specific stimulus-response curve, the additional search comprising:
stimulating said one or more peripheral nerves using a plurality of stimulus intensities to generate stimulus-response data points, wherein the plurality of stimulus intensities range from approximately thirty percent below to approximately thirty percent above the stimulation current corresponding to the maximal EP response amplitude;
determining peak-to-peak amplitudes of resulting EP response waveforms at each of the plurality of stimulus intensities;
rejecting the stimulus-response data points exhibiting SNR less than or equal to a predefined threshold and accepting the stimulus-response data points exhibiting SNR above the predefined threshold;
performing a linear regression over the accepted stimulus-response data points to construct a linear approximation of the patient-specific stimulus-response curve;
performing a nonlinear curve fit over the accepted stimulus-response data points to determine an optimal stimulation current corresponding to a maximal EP response amplitude; and
acquiring a high average-count EP response at the optimal stimulation current to establish an optimized EP baseline reference waveform for the patient.

8. The computer-implemented method of claim 7, wherein the additional search comprises acquiring between three and eight stimulus-response data points.

9. The computer-implemented method of claim 7, wherein the nonlinear curve fit is performed using a logistic model.

10. The computer-implemented method of claim 7, wherein the high average-count response acquired to establish the optimized EP baseline reference waveform comprises at least 100 average counts.

11. A system for determining a stimulus intensity and averaging count that elicit a maximal evoked potential (EP) response amplitude from a patient, the system comprising:
an EP stimulus optimizer comprising at least one stimulating electrode positioned in proximity to a peripheral nerve of the patient and one or more electrodes positioned on the patient's head in proximity to the sensory cortex;
a computing device in data communication with the EP stimulus optimizer and comprising at least one processor and a non-volatile memory for storing a plurality of instructions of programmatic code which, when executed, configure the processor to:
acquire neurological data, for the peripheral nerve of the patient, in the absence of peripheral stimulation to determine a baseline noise level;
compute, based on the baseline noise level, an initial count representing a function of a number of stimulus trials;
stimulate the peripheral nerve of the patient over the initial count using an initial stimulation current, wherein the initial stimulation current is selected from within a region of a modeled stimulus-response curve;
acquire EP response data over the initial count and determine an EP response amplitude associated with the initial stimulation current; and
determine whether the EP response amplitude is indicative of a valid EP response amplitude within the region,
wherein, if a valid EP response amplitude is not obtained then incrementally increase at least one of the initial count or the stimulation current and iterate the steps of acquiring EP response data and of determining if the EP response data is indicative of a valid EP response amplitude within the region, and
wherein, if either a valid EP response amplitude is obtained or predefined limits are reached on increasing the initial count or the stimulation current, terminate the execution of the plurality of instructions of programmatic code.

12. The system of claim 11, wherein the initial count represents a function of a number of stimulus trials to be averaged to achieve a predefined level of signal-to-noise ratio (SNR).

13. The system of claim 12, wherein the predefined level of signal-to-noise ratio is at least 2:1.

14. The system of claim 11, wherein the validity of the EP response amplitude is determined by performing a peak-to-peak calculation of the acquired EP response data using a peak detection algorithm.

15. The system of claim 11, wherein incrementally increasing the initial count comprises successively doubling the number of stimulus trials until the predefined level corresponding to the initial count is reached.

16. The system of claim 11, wherein the region corresponds to a segment of the modeled stimulus-response curve in which changes in stimulus current produce substantially linear changes in EP response amplitude.

17. The system of claim 11, wherein the plurality of instructions of programmatic code further configure the processor to perform an additional search to generate valid data points on a patient-specific stimulus-response curve, the additional search comprising:
stimulating said one or more peripheral nerves using a plurality of stimulus intensities to generate stimulus-response data points, wherein the plurality of stimulus intensities range from approximately thirty percent below to approximately thirty percent above the stimulation current corresponding to the maximal EP response amplitude;
determining peak-to-peak amplitudes of resulting EP response waveforms at each of the plurality of stimulus intensities;
rejecting the stimulus-response data points exhibiting SNR up to and including a predefined threshold and accepting the stimulus-response data points exhibiting SNR above the predefined threshold;
performing a linear regression over the accepted stimulus-response data points to construct a linear approximation of the patient-specific stimulus-response curve;
performing a nonlinear curve fit over the accepted stimulus-response data points to determine an optimal stimulation current corresponding to a maximal EP response amplitude; and
acquiring a high average-count EP response at the optimal stimulation current to establish an optimized EP baseline reference waveform for the patient.

18. The system of claim 17, wherein the additional search comprises acquiring between three and eight stimulus-response data points.

19. The system of claim 17, wherein the nonlinear curve fit is performed using a logistic model.

20. The system of claim 17, wherein the high average-count response acquired to establish the optimized EP baseline reference waveform comprises at least 100 average counts.
